(19) United States
(12) Patent Application Publication
(10) Pub. No.: US 2007/0049565 A1
(43) Pub. Date: Mar. 1, 2007

(54) Combination of cell necrosis inhibitor and lithium for treating neuronal death or neurological dysfunction

(75) Inventors: Byoung Joo Gwag, Suwon-si (KR); Young Ae Lee, Suwon-si (KR); Jin Hee Shin, Seoul (KR); Sung Ig Cho, Seoul (KR); Jae Sung Noh, Anyang-si (KR); Jae Young Cho, Suwon-si (KR); Ki Won Kim, Jeonju-si (KR); Hyang Ran Lim, Seoul (KR); Jae Keun Lee, Seoul (KR); Han Yeol Byun, Seongnam-si (KR)

Correspondence Address: SEED INTELLECTUAL PROPERTY LAW GROUP PLLC, 701 FIFTH AVE, SUITE 5400, SEATTLE, WA, 98104, US

(73) Assignee: Neurotech Pharmaceuticals Co., Ltd., Suwon-si (KR)

(21) Appl. No.: 11/503,379

(22) Filed: Aug. 11, 2006

Related U.S. Application Data
(60) Provisional application No. 60/780,245, filed on Mar. 8, 2006.

(30) Foreign Application Priority Data
Aug. 24, 2005 (KR) 10-2005-0078028

(51) Int. Cl. A61K 31/60; A61K 31/195; A61K 31/24; A61K 31/137

(52) U.S. Cl. 514/159; 514/534; 514/649; 514/567

(57) ABSTRACT

The present invention relates to a combination of cell necrosis inhibitor and lithium, process for the preparation of the combination, pharmaceutical formulation containing the combination and use of the combination by either concomitant or sequential administration for improvement of treatment of neuronal death or neurological dysfunction. The combination of the present invention shows a synergic effect and thus is useful for treating neurological diseases, such as amyotrophic lateral sclerosis (ALS, Lou Gehrig's disease), Alzheimer's disease, Parkinson's disease, Huntington's disease, stroke, traumatic brain injury or spinal cord injury; and for treating ocular diseases such as glaucoma, diabetic retinopathy or macular degeneration.

### CROSS-REFERENCE(S) TO RELATED APPLICATION(S)

This application claims the benefit under 35 U.S.C. § 119(e) of U.S. Provisional Patent Application No. 60/780,245 filed Mar. 8, 2006 and priority to South Korean Application No. 10-2005-0078028 filed Aug. 24, 2005; which applications are incorporated by reference herein in their entirety.

### BACKGROUND OF THE INVENTION

1. Field of the Invention

The present invention relates generally to a combination of a cell necrosis inhibitor and lithium, process for the preparation of the combination, pharmaceutical formulation containing the combination and use of the combination by either concomitant or sequential administration for improvement of treatment of neuronal death or neurological dysfunction. The combination of the present invention shows a synergic effect and thus is useful for treating neurological diseases such as amyotrophic lateral sclerosis (ALS, Lou Gehrig's disease), Alzheimer's disease, Parkinson's disease, Huntington's disease, stroke, traumatic brain injury or spinal cord injury, and ocular diseases such as glaucoma, diabetic retinopathy or macular degeneration.

2. Description of the Related Art

Neuronal death is a major neuropathological event in acute and chronic neurological diseases such as amyotrophic lateral sclerosis (ALS, Lou Gehrig's disease), Alzheimer's disease, Parkinson's disease, Huntington's disease, stroke, or spinal cord injury, and ocular diseases such as glaucoma, diabetic retinopathy or macular degeneration, and can result in catastrophic dysfunction in brain, spinal cord and eye (Osborne et al., 1999; Lewen et al., 2000; Danysz et al., 2001; and Behl et al., 2002). Thus, mechanisms and interventional therapy of neuronal death have been extensively studied.

A substantial body of evidence suggests that necrosis is a dominant pattern of pathological neuronal death and can be induced by activation of various intrinsic and extrinsic death pathways including oxidative stress and excitotoxicity (Beal, 1996; Dugan & Choi, 1994). Oxidative stress is described as excess accumulation of free radicals such as reactive oxygen or nitrogen species in cells due to a mismatch between generation and elimination of free radicals. Cellular overload of free radicals can attack target molecules including DNA, proteins, and lipids, which results in cell dysfunction and degeneration. Excitotoxicity is induced by excess activation of ionotropic glutamate receptors sensitive to N-methyl-D-aspartate (NMDA) and α-amino-3-hydroxy-5-methyl-4-isoxazolepropionic acid (AMPA). Oxidative stress and excitotoxicity cause cell body swelling, scattering condensation of nuclear chromatin, and early fenestration of plasma membrane, which results in cell necrosis (Gwag et al., 1997; Nicotera et al., 1997; Won et al., 2000).

Evidence has accumulated demonstrating that oxidative stress and excitotoxicity mediate neuronal death in animal models and patients of various neurological diseases (Rao & Weiss, 2003; Waldmeier, 2003; Meldrum, 2000). It includes mitochondrial abnormalities, generation of pro-oxidants, and oxidation of DNA, protein, and lipid in Alzheimer's disease (Mecocci et al., 1994), Parkinson's disease (Dauer et al., 2003), amyotrophic lateral sclerosis (ALS, Lou Gehrig's disease) (Beal, 2001), Huntington's disease (Beal et al., 1995), stroke (Won et al., 2002), spinal cord injury (Brown et al., 1992), and ocular diseases including glaucoma, diabetic retinopathy, and macular degeneration (Takahashi et al., 2004).

Several compounds preventing oxidative stress and excitotoxicity were shown to protect neurons in animal models of ALS (Andreassen et al., 2000; Gurney et al., 1997), Alzheimer's disease (Sung et al., 2004; Miguel-Hidalgo et al., 2002), stroke (Holtzman et al., 1996; Park et al., 1988), Huntington's disease (Andreassen et al., 2001; Beister et al., 2004), spinal cord injury (Faden & Salzman, 1992; Faden et al., 1994), Parkinson's disease (Prasad et al., 1999; Rabey et al., 1992), glaucoma (Neufeld et al., 2002; Pang et al., 1999), diabetic retinopathy (Chung et al., 2005; Smith et al., 2002), and macular degeneration (Richer et al., 2004).

Several compounds preventing oxidative stress and excitotoxicity have been examined for prevention of cell death and neurological function deficit in clinical trials of stroke, Alzheimer's disease, and Parkinson's disease (Gilgun-Sherki et al., 2002). However, the clinical trials of antioxidants such as vitamin E and acetyl-L-carnitine have failed to show beneficial effects in Alzheimer's disease and Parkinson's disease (Hudson & Tabet, 2003; Thal et al., 2003; Luchsinger et al., 2003; Morens et al., 1996). Low potency and blood brain barrier permeability of the antioxidants underlie unsuccessful outcome in the clinical trials (Gilgun-Sherki et al., 2002; Molina et al., 1997). A number of NMDA antagonists have been developed and shown to reduce hypoxic-ischemic brain injury in various animal models. However, none of them have been beneficial in the clinical trials of ischemic stroke patients mainly due to the narrow therapeutic index and time window of NMDA antagonists (Labiche et al., 2004; Hoyte et al., 2004; Ikonomidou. & Turski, 2002). Thus, the therapeutic limitation of necrosis-inhibiting compounds preventing oxidative stress and excitotoxicity remains to be resolved.

Apoptosis has been coined as an additional route of pathological neuronal death. Apoptosis is accompanied by cell body shrinkage, aggregated condensation of nuclear chromatin, and fenestration of nuclear membrane with preservation of plasma membrane (Kerr et al., 1972), which differs from neuronal cell necrosis showing cell body swelling, scattering condensation of nuclear chromatin, and collapse of plasma membrane with preservation of nuclear membrane (Gwag et al., 1995; Won et al., 2000).

Recently, neurotrophins that block neuronal apoptosis induce and/or potentiate neuronal cell necrosis in vitro and in vivo (Gwag & Kim, 2003; Koh et al., 1995; Won et al., 2000; Kim et al., 2002; and Barde 1994). This hints that apoptosis and necrosis may be propagated through mutually distinctive signaling pathways. Nuclear chromatin condensation, upregulation of pro-apoptotic proteins such as Bax, and activation of caspase-3, a downstream mediator of apoptosis, have been observed in human specimens of Alzheimer's disease (Kang et al., 2005; Su et al., 1997), Parkinson's disease (Hartman et al., 2000; Tatton, 2000), and ALS (Wootz et al., 2004, Biochem Biophys Res Commun., 322(1):281-6; Martin, 1999; Mu et al., 1996) and animal models of neurological diseases including Parkinson's disease (Turmel et al., 2001; Vila et al., 2001), ALS (Li et al, 2000; Gonzalez et al., 2000), stroke (Chan et al., 2004, Neurochem Res., 29(11):1943-9; Won et al., 2002; Choi, 1996), and traumatic spinal cord injury (Emery et al., 1998; Fiskum, 2000).

Anti-apoptosis drugs have been developed for the prevention of neuronal death. These include peptide inhibitors of caspases (Honig et al., 2000; Robertson et al., 2000), neurotrophic factors (Gwag & Kim, 2003; Lewin & Barde, 1996), and c-Jun N-terminal kinase (JNK) inhibitors such as CEP-1347 and CEP-11004 (Peng et al., 2004; Saporito et al., 2002). However, the therapeutic application of peptides, neurotrophic proteins, and JNK inhibitors should be compromised with transportation into brain (for example, peptides and proteins) and safety (for example, JNK inhibitors).

Recently, neuroprotective effects of lithium ion (Li⁺) have been reported in cultured neurons and in vivo (Kang et al., 2003; Chuang et al., 2002). Li⁺ is the lightest monovalent cation of the alkali metals, which was introduced into psychiatry in 1949 for the treatment of manic depressive illness and is widely used for the acute and prophylactic treatment of bipolar disorder and recurrent depression (Goodwin and Jamison, 1990). Li⁺ prevents neuronal apoptosis induced by low potassium (D'mello et al., 1994), ceramide (Centeno et al., 1998), staurosporine (Bijur et al., 2000), and beta amyloid (Ghribi et al., 2003) but does not attenuate cell necrosis-related neurotoxicity (Wie et al., Eur J Pharmacol. 2000; 392(3):117-23). Li⁺ prevents apoptosis by inducing expression of Bcl-2, an anti-apoptosis protein, and brain-derived neurotrophic factor and activating phosphoinositide 3-kinase (PI3-K)-phospholipase Cy pathway (Kang et al., 2003).

Accordingly, there is a need in the art for compositions and methods for treating neuronal death or neurological dysfunction. The present invention fulfills these needs and further provides other related advantages.

### BRIEF SUMMARY OF THE INVENTION

Groups of neuroprotective drugs that block neuronal cell necrosis induced by activation of NMDA receptor, free-radicals and/or zinc at submicromolar concentrations in cortical cell cultures and reduce infarct volume in animal models have been developed (See U.S. Pat. No. 6,964,982; No. 6,573,402; and No. 6,927,303, the disclosures of which are incorporated herein by reference in their entirety), and are used in the present invention.

Briefly stated, the present invention is based on surprising effects of a combination of (a) a cell necrosis inhibitor including, but is not limited to, the neuroprotective compounds disclosed by U.S. Pat. No. 6,964,982; No. 6,573,402; and No. 6,927,303, and (b) lithium or a pharmaceutically acceptable salt thereof. The combination of the present invention is more useful in neuroprotection and improving neurological function of acute and chronic neurological diseases than treatment with either agent alone.

Therefore, the present invention provides a method for treating neuronal death in neurological disease or ocular disease in a human or animal, which comprises administering to the human or animal in need thereof a therapeutically effective amount of cell necrosis inhibitor and concomitantly or sequentially administering a therapeutically effective amount of lithium or a pharmaceutically acceptable salt thereof.

The present invention also provides a single unit dosage form, a pharmaceutical formulation or a kit for treating neuronal death in neurological disease or ocular disease in a human or animal, which comprises a therapeutically effective amount of cell necrosis inhibitor and a therapeutically effective amount of lithium or a pharmaceutical acceptable salt thereof.

Preferably, the present invention provides the method, the single unit dosage form, the pharmaceutical formulation, or the kit, wherein the neurological disease is any one selected from amyotrophic lateral sclerosis (ALS, Lou Gehrig's disease), Alzheimer's disease, Parkinson's disease, Huntington's disease, stroke, traumatic brain injury, and spinal cord injury.

Preferably, the present invention provides the method, the single unit dosage form, the pharmaceutical formulation, or the kit, wherein the ocular disease is any one selected from glaucoma, diabetic retinopathy and macular degeneration.

Preferably, the present invention provides the method, the single unit dosage form, the pharmaceutical formulation, or the kit, wherein the cell necrosis inhibitor is at least one selected from:

(i) benzylaminosalicylic acid derivatives of the following formula (I) or pharmaceutically acceptable salts thereof, and

(ii) tetrafluorobenzyl derivatives of the following formula (II) or pharmaceutically acceptable salts thereof: 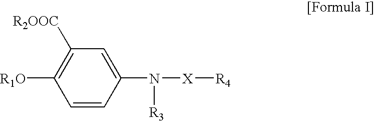
wherein,

X is CO, SO₂ or (CH₂)ₙ, wherein n is an integer from 1 to 5;

R₁ is hydrogen, alkyl or alkanoyl;

R₂ is hydrogen or alkyl;

R₃ is hydrogen or an acetoxy group; and

R₄ is a phenyl group which is unsubstituted or substituted with one or more of nitro, halogen, haloalkyl, and C₁-C₅ alkoxy; 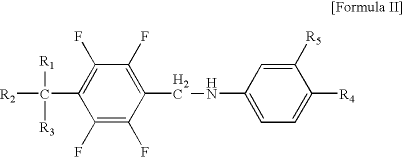
wherein,

R₁, R₂ and R₃ are independently hydrogen or halogen;

R₄ is hydroxy, alkyl, alkoxy, halogen, alkoxy substituted with halogen, alkanoyloxy or nitro; and

R₅ is carboxyl acid, ester having C₁-C₄ alkyl, carboxyamide, sulfonic acid, halogen or nitro.

These and other aspects of the present invention will become apparent upon reference to the following detailed description and attached drawings. All references disclosed herein are hereby incorporated by reference in their entirety as if each was incorporated individually.

### BRIEF DESCRIPTION OF THE DRAWINGS

The above and other objects, features and other advantages of the present invention will be more clearly understood from the following detailed description taken in conjunction with the accompanying drawings, in which:

FIG. 1. The effects of vitamin E, 2-hydroxy-TTBA, 2-hydroxy-TPEA, and Li⁺ against free radical-mediated neuronal cell necrosis in cortical cell cultures:

A: The effects of vitamin E, 2-hydroxy-5-(2,3,5,6-tetrafluoro-4-trifluoromethyl-benzylamino)-benzoic acid (hereinafter, “2-hydroxy-TTBA”), and 2-hydroxy-5-(2-(4-trifluoromethylphenyl)ethylamino)-benzoic acid (hereinafter, “2-hydroxy-TPEA”) on Fe²⁺-induced neurotoxicity.

Mouse cortical cell cultures (DIV 11-15) were exposed to 50 μM Fe²⁺, alone or with indicated doses of 2-Hydroxy-TTBA, 2-Hydroxy-TPEA, or Vitamin E. Neuronal death was analyzed 24 hr later by measuring levels of LDH released into the bathing medium, mean±SEM (n=9-12 culture wells per condition), scaled to mean LDH efflux value 24 hr after sham wash (=0) and continuous exposure to 500 μM NMDA (=100). *, Significant difference from Fe²⁺ alone, p<0.05 using ANOVA and Student-Newman-Keuls test.

B: The effects of 2-hydroxy-TTBA and 2-hydroxy-TPEA on DL-buthionine-[S,R]-sulfoximine (a glutathione-depleting agent, hereinafter “BSO”)-induced neurotoxicity.

Mouse cortical cell cultures (DIV 11-15) were exposed to 10 mM BSO, alone or with indicated doses of 2-Hydroxy-TTBA or 2-Hydroxy-TPEA. Neuronal death was analyzed 24 hr later by measuring levels of LDH released into the bathing medium, mean±SEM (n=9-12 culture wells per condition). *, Significant difference from BSO alone, p<0.05 using ANOVA and Student-Newman-Keuls test.

C: Li⁺ does not attenuate free radical neurotoxicity.

Mouse cortical cell cultures (DIV 11-15) were exposed to 50 μM Fe²⁺ or 10 mM BSO, alone or with inclusion of 5 mM Li⁺. Neuronal death was analyzed 24 hr later by measuring levels of LDH released into the bathing medium, mean±SEM (n=9-12 culture wells per condition).

FIG. 2. The effects of vitamin E, 2-hydroxy-TTBA, 2-hydroxy-TPEA, and Li⁺ against neuronal cell apoptosis in cortical cell cultures:

A: The neuroprotective effects of Li⁺ against calyculin A or cyclosporine A-induced neuronal apoptosis.

Mouse cortical cell cultures (DIV 10-12) were exposed to 20 μM cyclosporine A or 10 nM calculin A, alone or with inclusion of 0.3-30 mM Li. Neuronal death was analyzed 24-28 hr later by measuring levels of LDH released into the bathing medium, mean±SEM (n=9-12 culture wells per condition).

B: Vitamin E, 2-hydroxy-TTBA, and 2-hydroxy-TPEA do not attenuate neuronal cell apoptosis.

Mouse cortical cell cultures (DIV 10-12) were exposed to 20 μM cyclosporine A, alone or with 100 μM Vitamin E, 1 μM 2-hydroxy-TTBA, or 1 μM 2-hydroxy-TPEA. Neuronal death was analyzed 24 hr later by measuring levels of LDH released into the bathing medium, mean±SEM (n=9-12 culture wells per condition).

FIG. 3. Analysis of oxidative stress and neuronal death in the lumbar spinal cord from ALS transgenic mice (G93AA):

A: The fluorescent photomicrographs of the lumbar spinal cord section immunolabeled with nitrotyrosine antibody (green, top panel) or double-labeled (bottom panel) with MitoTracker CM-H2XRos (red) and NeuN antibody (neuronal marker, green) in wild type (a,c) or ALS transgenic mice (b,d) at ages of 8 week. Arrows indicate motor neurons.

B: The fluorescence intensity of nitrotyrosine was analyzed in the ventral motor neurons at ages of 4 to 14 weeks, mean±SEM (n=25 sections from five mice per each group). * Significant difference between wild type and ALS transgenic mice at the same age, using Independent-Samples t-test.

C: Degeneration of the spinal motor neurons from ALS transgenic mice.

The number of the viable motor neurons in the lumbar ventral horn was analyzed after staining with cresyl violet at indicated points of age, mean±SEM (n=5 mice per each group).

FIG. 4. Activation of Fas-mediated apoptosis pathways in ALS transgenic mice:

A: Western blot analysis showing expression of Fas, FADD, and actin in the lumbar segment from wild type [Tg(−)] or ALS transgenic mice [Tg(+)] at indicated ages (top panel). Bottom panel shows interaction of Fas and FADD using Western blot analysis of FADD antibody following immunoprecipitation with Fas antibody in the same samples above.

B: Bright-field photomicrographs of the spinal motor neurons taken after immunolabeling with Fas antibody from Tg(−) (a) or Tg(+) (b) at age of 12 weeks.

C: Western blot analysis showing expression of caspase 8, caspase 3, and actin in the lumbar segment from Tg(−) or Tg(+) at indicated points of age.

D: Fluorescence photomicrographs of the lumbar ventral sections taken after immunolabeling with an antibody for cleaved caspase 3 from Tg(−) (a) or Tg(+) (b) at age of 12 weeks.

FIG. 5. Oxidative stress and apoptosis in the spinal motor neurons from ALS transgenic mice: effects of 2-hydroxy TTBA and Li⁺:

A: 2-hydroxy TTBA, but not Li⁺, prevents oxidative stress. Mouse cortical cell cultures (DIV 11-15) were exposed to 30 μM Fe²⁺ or 10 mM BSO, alone or in the presence of 1 μM 2-hydroxy TTBA, 10-100 μM vitamin E, or 5 mM Li⁺. Neuronal death was analyzed 24 h later by measuring LDH efflux in the bathing media (mean±SEM, n=12). *, Significant difference from the relevant control (Fe²⁺ or BSO alone), p<0.05 using ANOVA and Student-Newman-Keuls test.

B & C: Li⁺, but not 2-hydroxy TTBA, prevents apoptosis.

(B) Neuron-rich cortical cell cultures (DIV 7) were deprived of serum, alone or with addition of 100 μM zVADfmk, 1 μM 2-hydroxy TTBA, or 5 mM Li⁺. Neuronal death was analyzed 24 hr later by counting viable neurons excluding trypan (mean±SEM, n=4). *, Significant difference from the relevant control (serum deprivation alone), p<0.05 using ANOVA and Student-Neuman-Keuls test. (C) Western blot analysis of FADD antibody following immunoprecipitation with Fas antibody in the same samples above.

D: Fluorescent photomicrographs of the lumbar ventral section immunolabeled with nitrotyrosine antibody from the wild type (a), or ALS transgenic mice treated with vehicle (b), or 2-hydroxy TTBA for 2 weeks starting from at age of 8 weeks (c). Arrows indicate motor neurons (top panel). Levels of nitrotyrosine were quantitated, mean±SEM (n=15 sections from 3 mice per each condition (bottom panel). p<0.05 using ANOVA and Student-Newman-Keuls test.

E: Same as D except measurement of the fluorescence intensity of oxidized MT red CM-H2XRos. p<0.05 using ANOVA and Student-Newman-Keuls test.

F: Western blot analysis of Fas, FADD, cleaved caspase-8, cleaved caspase-3, and actin in the lumbar segment from the wild type [Tg(−)] or Tg(+) treated with saline, 2-hydroxy TTBA, or Li⁺ for 4 weeks starting from age of 8 weeks.

FIG. 6. Co-administration of 2-hydroxy TTBA and Li⁺ synergistically improves motor function in ALS transgenic mice:

Animals were daily fed with 2-hydroxy TTBA (30 mg/kg/d), 0.2% lithium carbonate (200 mg/kg/d, Li), or a combination of both (2-hydroxy TTBA+Li) from 8 weeks of age in diet. Body weight (A), extension reflex (B), PaGE test (C), and Rotarod test (D) were analyzed at the indicated points of age, mean±SEM (n=13 per each group) *, p<0.05 compared to the vehicle; #, p<0.05 between 2-hydroxy TTBA (or Li) alone and combination of 2-hydroxy TTBA and Li.

FIG. 7. Co-administration of 2-hydroxy TTBA and Lithium synergistically delays onset of motor function deficit, survival, and degeneration of the motor neurons in ALS mice:

Animals were daily fed with 2-hydroxy TTBA (30 mg/kg/d), 0.2% lithium carbonate (Li), or a combination of both (2-hydroxy TTBA+Li) from 8 weeks of age in diet.

A & B: Cumulative probability of onset of motor function deficits (A) and cumulative probability of survival (B) in ALS transgenic mice.

C & D: (C) Bright-field photomicrographs of cresyl violet-stained ventral horn sections from the wild type (a), or G93AA transgenic mice treated with vehicle (b), or with a combination of 2-hydroxy TTBA+Li (c) at 16 weeks of age. (D) The number of viable motor neurons in the lumbar ventral horn was analyzed at 16 weeks of age, mean±SEM (n=20 sections from four mice per each group) *, p<0.01 compared to the vehicle; #, p<0.01 between 2-hydroxy TTBA (or Li) alone and combination of 2-hydroxy TTBA and Li, using ANOVA and Student-Newman-Keuls test.

FIG. 8. The effect of 2-hydroxy-TPEA and lithium on amyloid beta production in TAPP transgenic mice:

A: The fluorescent photomicrographs of brain sections stained with 1% thioflavine-S from 14.5 month-old wild type (a) and 14.5 month-old TAPP transgenic mice (b), treatment with 25 mg/kg/day of 2-hydroxy-TPEA (c), or co-treatment with 25 mg/kg/day of 2-hydroxy-TPEA and 300 mg/kg/day of lithium carbonate (d).

B: The fluorescence intensity of thioflavin-S was analyzed in the brain sections from 14.5 month-old TAPP transgenic mice (control), treatment with 25 mg/kg/day of 2-hydroxy-TPEA (2-hydroxy-TPEA), or co-treatment with 25 mg/kg/day of 2-hydroxy-TPEA and 300 mg/kg/day of lithium carbonate (2-hydroxy-TPEA+Li), mean±SEM (n=16 serial sections from two mice per each group). *, Significant difference from control, p<0.05 using ANOVA and Student-Newman-Keuls test.

C: The SDS-insoluble Aβ42 levels were analyzed by calorimetric sandwich ELISA kit in the brain homogenates from 14.5 months old TAPP transgenic mice (control) and treatment with 25 mg/kg/day of 2-hydroxy-TPEA (2-hydroxy-TPEA), or 25 mg/kg/day of 2-hydroxy-TPEA+300 mg/kg/day of lithium carbonate (2-hydroxy-TPEA+Li), mean±SEM (n=4 animals). *, Significant difference from control p<0.05 using ANOVA and Student-Newman-Keuls test.

### DETAILED DESCRIPTION OF THE INVENTION

The present invention relates to a combination of at least one cell necrosis inhibitor; and lithium or a pharmaceutically acceptable salt thereof. The present invention also relates to a method for improving the treatment of neuronal death in neurological disease or ocular disease, a single unit dosage form, a pharmaceutical formulation or a kit using the combination.

Therefore, the present invention provides a method for treating neuronal death in neurological disease or ocular disease in a human or animal, which comprises administering to the human or animal in need thereof a therapeutically effective amount of a cell necrosis inhibitor and concomitantly or sequentially administering a therapeutically effective amount of lithium or a pharmaceutically acceptable salt thereof.

Examples of neurological diseases that may be treated with the combination of the present invention include, but are not limited to, amyotrophic lateral sclerosis (ALS, Lou Gehrig's disease), Alzheimer's disease, Parkinson's disease, Huntington's disease, stroke, traumatic brain injury, and spinal cord injury. Examples of ocular diseases that may be treated with the combination of the present invention include, but are not limited to, glaucoma, diabetic retinopathy and macular degeneration. Relations between the concrete diseases mentioned above and the combination of the present invention are described below in more detail.

The combination of the present invention comprises a cell necrosis inhibitor and, preferably, the cell necrosis inhibitor is, but is not limited to, at least one selected from:

(i) benzylaminosalicylic acid derivatives of the following formula (I) or pharmaceutically acceptable salts thereof, and

(ii) tetrafluorobenzyl derivatives of the following formula (II) or pharmaceutically acceptable salts thereof: 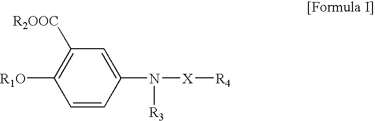
wherein,

X is CO, SO₂ or (CH₂)ₙ, wherein n is an integer from 1 to 5;

R₁ is hydrogen, alkyl or alkanoyl;

R₂ is hydrogen or alkyl;

R₃ is hydrogen or an acetoxy group; and

R₄ is a phenyl group which is unsubstituted or substituted with one or more of nitro, halogen, haloalkyl, and C₁-C₅ alkoxy; 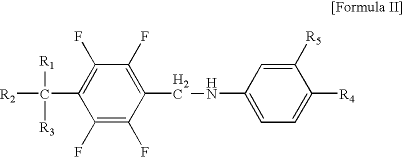
wherein,

R₁, R₂ and R₃ are independently hydrogen or halogen;

R₄ is hydroxy, alkyl, alkoxy, halogen, alkoxy substituted with halogen, alkanoyloxy or nitro; and

R₅ is carboxyl acid, ester having C₁-C₄ alkyl, carboxyamide, sulfonic acid, halogen or nitro.

In formula I and II, alkyl is C₁-C₄ alkyl, and more preferably C₁-C₂ alkyl. Alkyl described above includes, but is not limited to, methyl, ethyl, propyl, isopropyl, n-butyl, sec-butyl, and tert-butyl. Alkoxy is C₁-C₄ alkoxy, and more preferably C₁-C₂ alkoxy. Alkoxy described above includes, but is not limited to, methoxy, ethoxy, and propanoxy. Halogen includes, but is not limited to, fluoride, chloride, bromide, and iodide. Alkanoyl is C₂-C₁₀ alkanoyl, and more preferably C₃-C₅ alkanoyl. Alkanoyl described above includes, but is not limited to, ethanoyl, propanoyl, and cyclohexanecarbonyl. Alkanoyloxy is C₂-C₁₀ alkanoyloxy, and more preferably C₃-C₅ alkanoyloxy. Alkanoyloxy described above includes, but is not limited to, ethanoyloxy, propanoyloxy, and cyclohexanecarbonyloxy.

The benzylaminosalicylic acid derivatives and tetrafluorobenzyl derivatives are more preferable than other cell necrosis inhibitors when considering their efficacy and synergic effect with lithium. These cell necrosis inhibitors (See U.S. Pat. No. 6,964,982; No. 6,573,402; and No. 6,927,303, the disclosures of which are incorporated herein by reference in their entirety) in nanomolar range block completely cell-necrosis-related neurotoxicity and confirm neuroprotective effects in animal models of stroke, spinal cord injury or ALS.

After considering safety and therapeutic efficiency including neuroprotective effect of cell necrosis inhibitors, and combination synergy with lithium, examples of the benzylaminosalicylic acid derivatives include, but are not limited to,

5-benzylaminosalicylic acid (BAS),

5-(4-nitrobenzyl)aminosalicylic acid (NBAS),

(5-(4-chlorobenzyl)aminosalicylic acid (CBAS),

(5-(4-trifluoro-methylbenzyl)aminosalicylic acid (TBAS),

(5-(4-fluorobenzyl)aminosalicylic acid (FBAS),

5-(4-methoxybenzyl)aminosalicylic acid (MBAS),

5-(pentafluoro-benzyl)aminosalicylic acid (PBAS),

5-(4-nitrobenzyl)amino-2-hydroxy ethylbenzoate,

5-(4-nitrobenzyl)-N-acetylamino-2-hydroxy ethylbenzoate,

5-(4-nitrobenzyl)-N-acetylamino-2-acetoxy ethylbenzoate,

5-(4-nitrobenzoyl)aminosalicylic acid,

5-(4-nitrobenzenesulfonyl)aminosalicylic acid,

5-[2-(4-nitrophenyl)-ethyl]aminosalicylic acid (NPAA),

5-[3-(4-nitrophenyl)-n-propyl]aminosalicylic acid (NPPAA),

2-hydroxy-5-(2-(4-trifluoromethyl-phenyl)ethylamino)-benzoic acid (2-hydroxy-TPEA), and

pharmaceutically acceptable salts thereof;

and examples of the tetrafluorobenzyl derivatives include, but are not limited to,

2-hydroxy-5-(2,3,5,6-tetrafluoro-4-trifluoromethyl-benzylamino)-benzoic acid (2-Hydroxy-TTBA),

2-nitro-5-(2,3,5,6-tetrafluoro-4-trifluoromethyl-benzylamino)benzoic acid,

2-chloro-5-(2,3,5,6-tetrafluoro-4-trifluoromethylbenzylamino)benzoic acid,

2-bromo-5-(2,3,5,6-tetrafluoro-4-trifluoromethyl-benzylamino)benzoic acid,

2-hydroxy-5-(2,3,5,6-tetrafluoro-4-methylbenzylamino)benzoic acid,

2-methyl-5-(2,3,5,6-tetrafluoro-4-trifluoromethyl-benzylamino)benzoic acid,

2-methoxy-5-(2,3,5,6-tetrafluoro-4-trifluoromethylbenzylamino)benzoic acid,

5-(2,3,5,6-tetrafluoro-4-trifluoromethyl-benzylamino)-2-trifluoromethoxy benzoic acid,

2-nitro-4-(2,3,5,6-tetrafluoro-4-trifluoromethylbenzylamino)phenol,

2-chloro-4-(2,3,5,6-tetrafluoro-4-trifluoromethylbenzylamino)phenol,

2-hydroxy-5-(2,3,5,6-tetrafluoro-4-trifluoromethyl-benzylamino)benzamide,

2-hydroxy-5-(2,3,5,6-tetrafluoro-4-trifluoromethylbenzylamino)benzenesulfonic acid,

methyl 2-hydroxy-5-(2,3,5,6-tetrafluoro-4-trifluoromethyl-benzylamino)benzoate,

2-ethanoyloxy-5-(2,3,5,6-tetrafluoro-4-trifluoromethyl-benzylamino)benzoic acid,

2-propanoyloxy-5-(2,3,5,6-tetrafluoro-4-trifluoromethylbenzylamino)benzoic acid,

2-cyclohexanecarbonyloxy-5-(2,3,5,6-tetrafluoro-4-trifluoromethylbenzylamino)benzoic acid, and

pharmaceutically acceptable salts thereof.

The cell necrosis inhibitor compounds of the present invention can exist as a pharmaceutically acceptable salt. Pharmaceutically acceptable acid addition salts of the present compounds can be formed of the compound itself, or of any of its esters, and include the pharmaceutically acceptable salts which are often used in pharmaceutical chemistry. For example, salts may be formed with organic or inorganic acids. Suitable organic acids include maleic, fumaric, benzoic, ascorbic, succinic, methanesulfonic, benzenesulfonic, toluenesulfonic, acetic, oxalic, trifluoroacetic, propionic, tartaric, salicylic, citric, gluconic, lactic, mandelic, cinnamic, aspartic, stearic, palmitic, formic, glycolic, glutamic, and benzenesulfonic acids. Suitable inorganic acids include hydrochloric, hydrobromic, sulfuric, phosphoric, and nitric acids. Additional salts include chloride, bromide, iodide, bisulfate, acid phosphate, isonicotinate, lactate, acid citrate, oleate, tannate, pantothenate, bitartrate, gentisinate, gluconate, glucaronate, saccharate, ethanesulfonate, p-toluenesulfonate, and pamoate (i.e., 1,1′-methylene-bis-(2-hydroxy-3-naphthoate)) salts. The term “pharmaceutically acceptable salt” is intended to encompass any and all acceptable salt forms.

Pharmaceutically acceptable salts can be formed by conventional and known techniques, such as by reacting an inhibitor compound of this invention with a suitable acid as disclosed above. Such salts are typically formed in high yields at moderate temperatures, and often are prepared by merely isolating the compound from a suitable acidic wash in the final step of the synthesis. The salt-forming acid may be dissolved in an appropriate organic solvent, or aqueous organic solvent, such as an alkanol, ketone or ester. On the other hand, if the compound of the present invention is desired in the free base form, it may be isolated from a basic final wash step, according to known techniques. For example, a typical technique for preparing a hydrochloride salt is to dissolve the free base in a suitable solvent, and dry the solution thoroughly, as over molecular sieves, before bubbling hydrogen chloride gas through it.

In addition, some of the cell necrosis inhibitor compounds of the present invention may be in a hydrated form, and may exist as solvated or unsolvated form. A part of compounds exist as crystal form or amorphous form, and any physical form is included in the scope of the present invention.

The cell necrosis inhibitors of the present invention may contain one or more asymmetric carbon atoms and therefore exist in two or more stereoisomeric forms. The present invention includes these individual stereoisomers of the inhibitors of the present invention.

The combination of the present invention comprises lithium or a pharmaceutically acceptable salt thereof and, preferably, the salt includes, but is not limited to, lithium carbonate, lithium chloride, lithium bromide, lithium acetate, lithium citrate, lithium succinate, lithium acetylsalicylate, lithium benzoate, lithium bitartrate, lithium nitrate, lithium selenate, lithium sulphate, lithium aspartate, lithium gluconate and lithium thenoate.

In addition, the combination of the present invention may comprise a lithium salt of the benzylaminosalicylic acid derivative or the tetrafluorobenzyl derivative.

Further, the present invention provides a single unit dosage form, a pharmaceutical formulation or a kit comprising the cell necrosis inhibitor and lithium or its salt. A kit may also include instructions.

The combination of the present invention may be produced in one pharmaceutical formulation comprising both the cell necrosis inhibitor and lithium (or its salt) or in two different pharmaceutical formulations, one for the cell necrosis inhibitor and one for the lithium. The pharmaceutical formulation may be in the form of tablets, capsules, powders, mixtures, solutions, suspensions or other suitable pharmaceutical formulation forms. The pharmaceutical formulation of the present invention may comprise a pharmaceutically acceptable excipient for easiness of manufacturing, and appearance and stability of the formulation.

Routes of administration of the combination of the present invention include, but are not limited to, oral, topical, subcutaneous, transdermal, subdermal, intramuscular, intra-peritoneal, intravesical, intra-articular, intra-arterial, intra-venous, intra-dermal, intra-cranial, intra-lesional, intra-tumoral, intra-ocular, intra-pulmonary, intra-spinal, intraprostatic, placement within cavities of the body, nasal inhalation, pulmonary inhalation, impression into skin and electrocorporation.

To produce pharmaceutical formulations of the combination of the invention in the form of dosage units for oral application, the selected compounds may be mixed with a solid excipient, for example, a diluent such as lactose, mannitol, microcrystalline cellulose and corn starch; a binder such as gelatin and polyvinylpyrrolidone; a disintegrator such as sodium starch glycolate and cross-carmellose sodium; a lubricant such as magnesium stearate, wax and so on; and the like, and then compressed into tablets. If coated tablets are required, the tablet cores prepared above may be coated with a coating material such as gelatin, hydroxypropylmethylcellulose and so on.

For the formulation of soft gelatin capsules, the two active substances may be admixed with, for example, a vegetable oil or poly-ethylene glycol. Hard gelatin capsules may contain granules of the two active substances using a method well known to those skilled in the art.

Liquid formulation for oral application may be in the form of syrups, solutions or suspensions, and such liquid formulations may contain coloring agents, flavoring agents, sugar, stabilizers, surfactants, thickening agent or other excipients known to those skilled in the art.

Solutions for parenteral applications by injection can be prepared in an aqueous solution of a water-soluble pharmaceutically acceptable salt of the two active ingredients, preferably in a concentration of from about 0.1% to about 20% by weight. These solutions may also contain stabilizing agents, buffering agents and/or pH-adjusting agents, and may be conveniently prepared by conventional methods.

Further, the present invention provides a kit comprising the combination of the cell necrosis inhibitor and lithium or a pharmaceutically acceptable salt thereof, optionally with instructions for use.

The particular therapeutic agent administered, the amount per dose, the dose schedule and the route of administration should be decided by the practitioner using methods known to those skilled in the art and will depend on the type of neurological disease or ocular disease, the severity of the diseases, the location of the diseases and other clinical factors such as the size, weight and physical condition of the recipient. In addition, in vitro assays may optionally be employed to help identify optimal ranges for sequence administration.

For the purpose of this invention, daily dosage of the cell necrosis inhibitor may be in the range of about 0.1 mg-100 g/kg bodyweight, preferably about 0.5 mg-10 g/kg bodyweight, more preferably about 1 mg-1 g/kg bodyweight. Also, daily dosage of lithium for the adult human may generally be in the range of 1-2000 mg, preferably 20-600 mg, more preferably 50-600 mg/kg bodyweight (See U.S. Pat. No. 4,753,964, the disclosure of which is incorporated herein by reference in its entirety). As occasion demands, the combination of the present invention can be administered in small doses 1 to 4 times a day over variable times from weeks to months.

Hereinafter, embodiments of the present invention are described in considerable detail to help those skilled in the art further understand the present disclosure. However, the following examples are offered by way of illustration and are not intended to limit the scope of the invention. It is apparent that various changes may be made without departing from the spirit and scope of the invention or sacrificing all of its material advantages.

### EXAMPLE 1

### Mixed Cortical Cell Cultures of Neurons and Glia

For mixed neuron-glia culture, mouse cerebral cortices were removed from brains of the 11-15 day-old-fetal mice (E11-15), gently triturated and plated on 24 well plates (2×10⁵ cells/plate) precoated with 100 μg/ml poly-D-lysine and 4 μg/ml laminin. Cultures were maintained at 37° C. in a humidified 5% CO₂ atmosphere. Plating media consist of Eagles minimal essential media (MEM, Earles salts, supplied glutamine-free) supplemented with 5% horse serum, 5% fetal bovine serum, 26.5 mM bicarbonate, 2 mM glutamine, and 21 mM glucose.

After 7-8 days in vitro (DIV 7-8), 10 μM cytosine arabinofuranoside (Ara-C) was included to halt overgrowth of glia. The drug treatment was carried on DIV 11-15 cortical cell culture. Overall neuronal cell injury was assessed by measuring amount of lactate dehydrogenase (LDH) released into the bathing medium 24 hr after neurotoxic insults as previously described (Koh and Choi, J Neurosci Methods 20:83-90, 1987).

### EXAMPLE 2

### Blockade of Free Radical Neurotoxicity by Vitamin E, Trolox, 2-Hydroxy-TTBA, 2-Hydroxy-TPEA, BAS, NBAS, CBAS, MBAS, FBAS, PBAS, NPM, NPPAA and TBAS

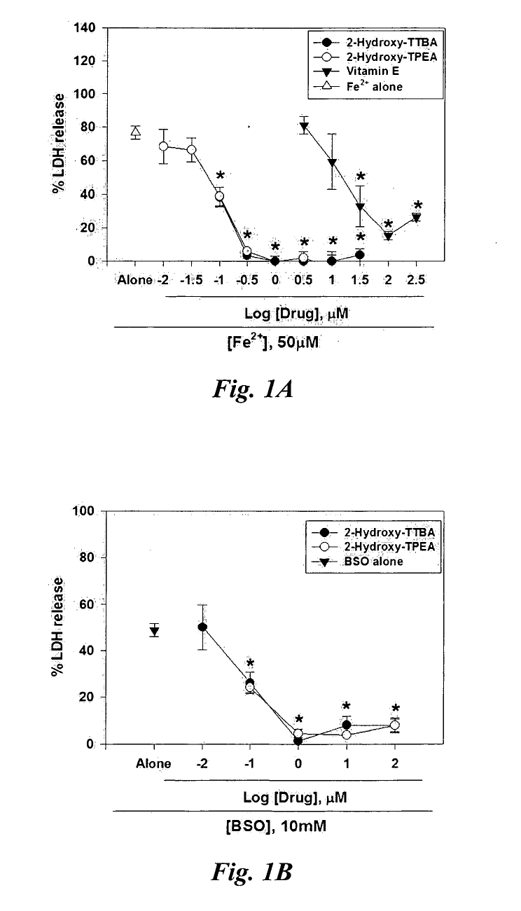

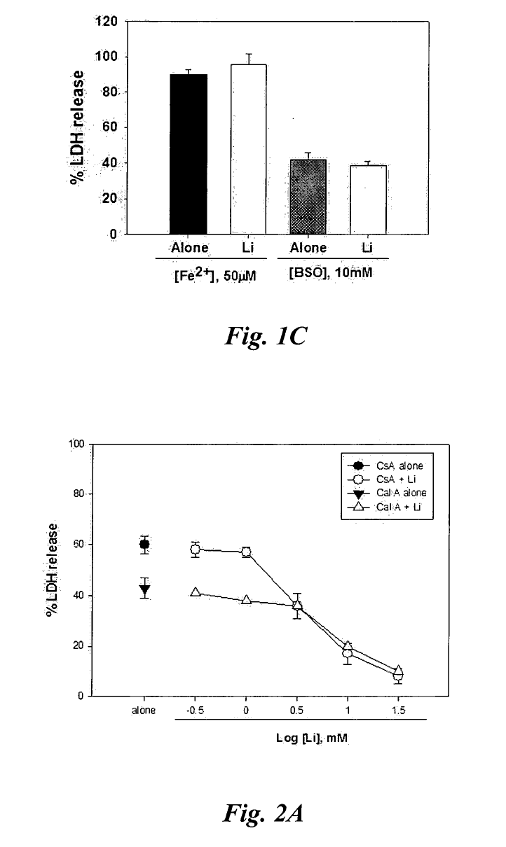

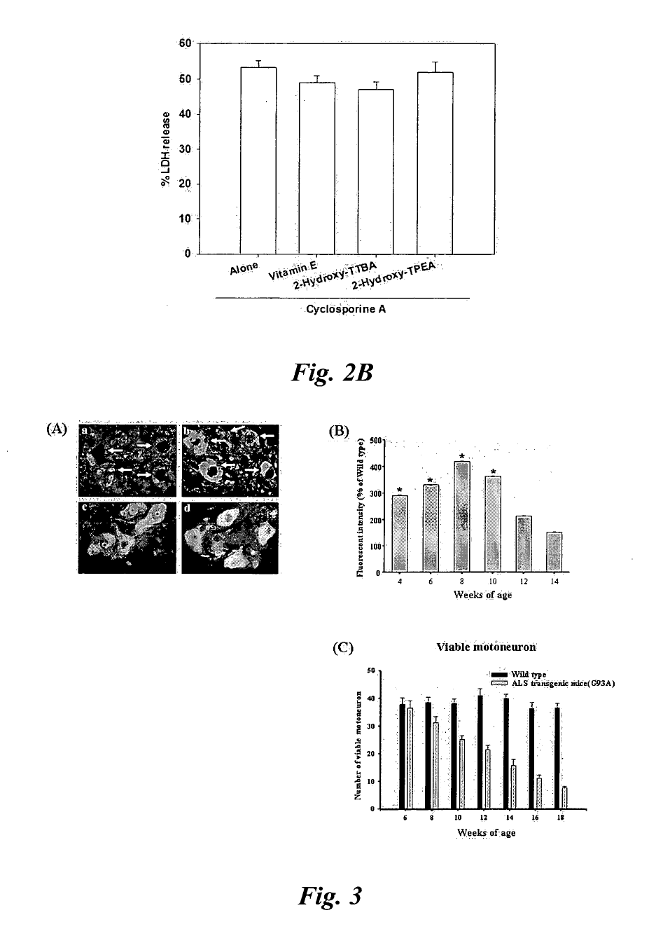

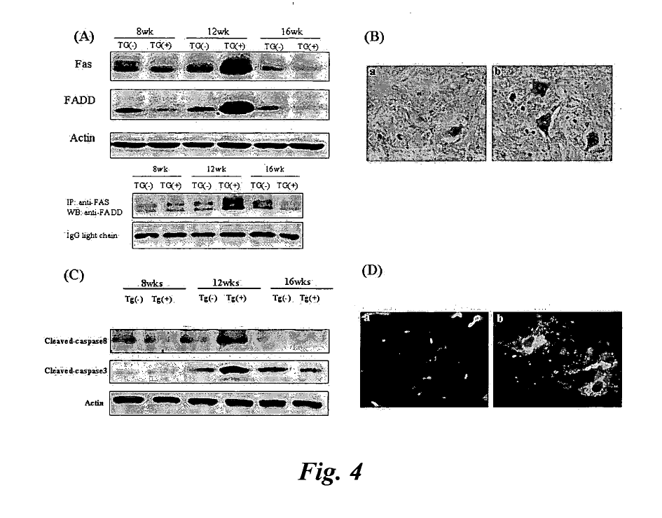

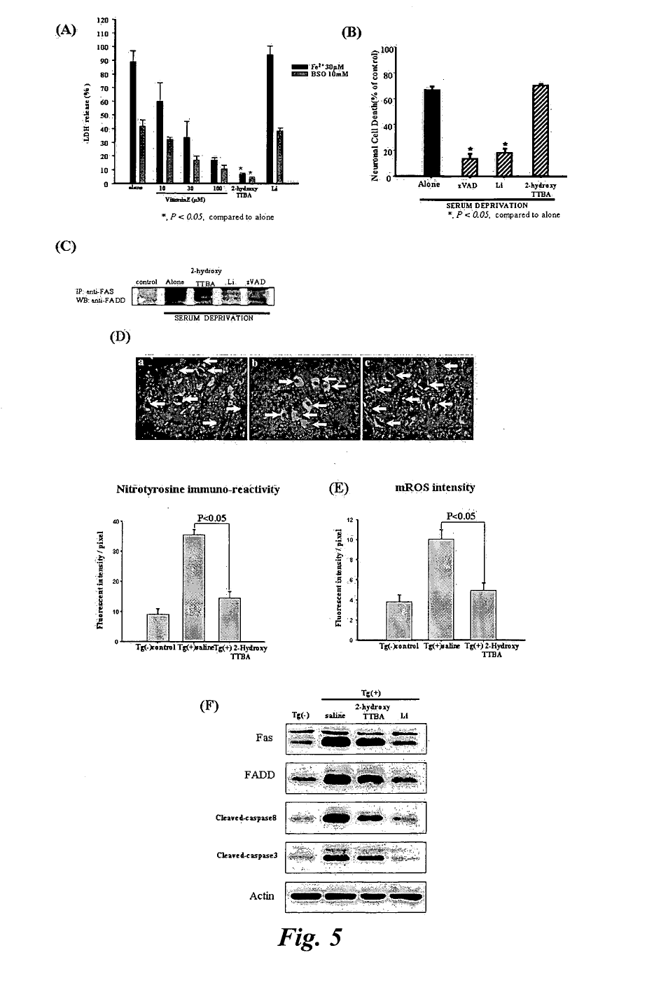

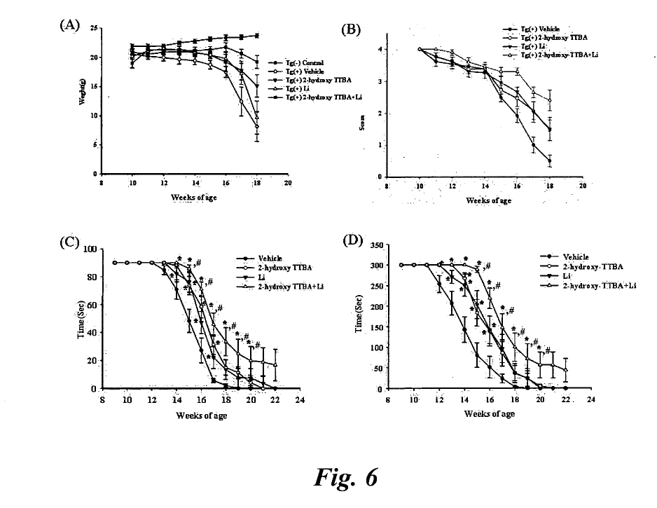

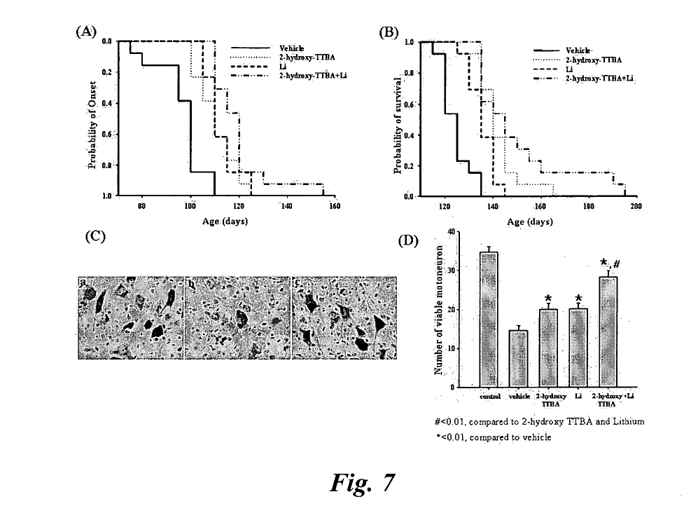

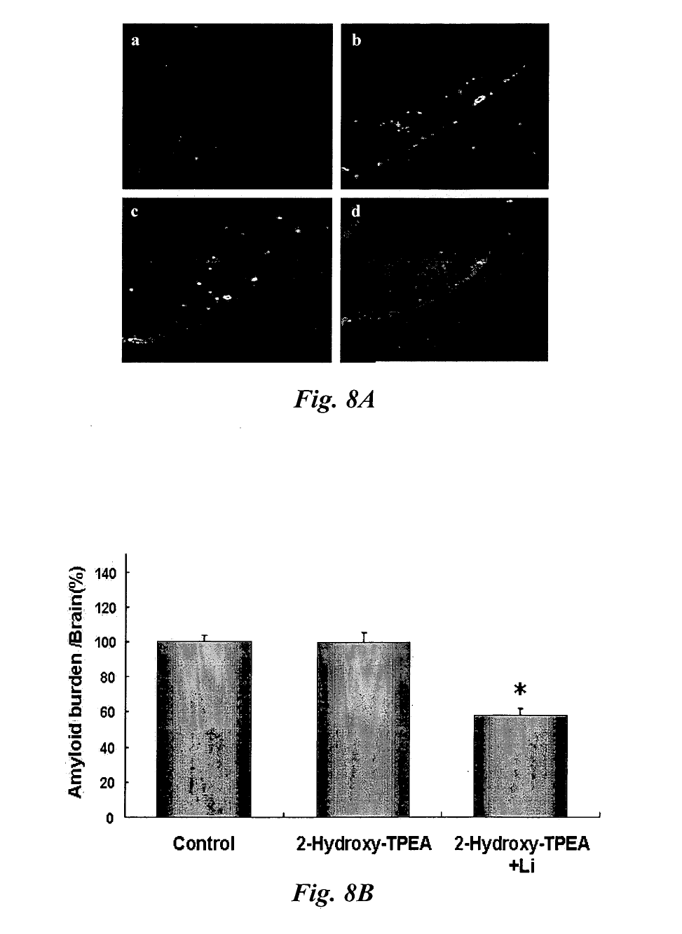

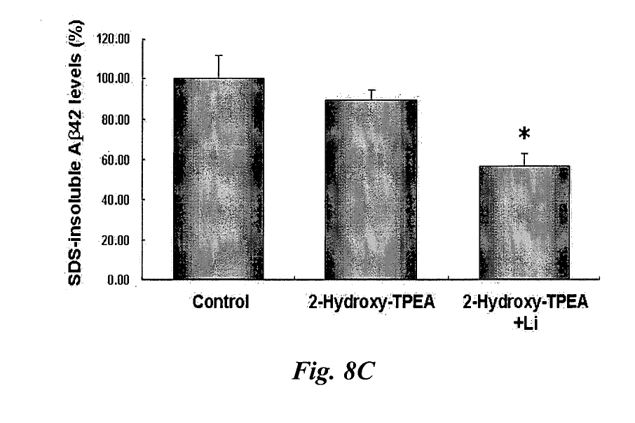

Oxidative stress was induced by exposing mixed cortical cell cultures containing neurons and glia (DIV 11-15) to 50 μM FeCl₂, a hydroxyl radical-producing transition metal via a Fenton reaction, or 10 mM DL-buthionine-[S,R]-sulfoximine (BSO), a glutathione depleting agent. Widespread neuronal death was observed 24 hours later. Concurrent administration of 2-Hydroxy-TTBA or 2-Hydroxy-TPEA nearly completely blocked free radical neurotoxicity at doses as low as 0.3 μM (FIGS. 1A & 1B). Administration of vitamin E prevented Fe²⁺-induced free radical neurotoxicity at higher doses. This implies that 2-Hydroxy-TTBA or 2-Hydroxy-TPEA is a potent neuroprotectant against oxidative stress. Neuroprotective effects of several cell necrosis inhibitors were analyzed as IC₅₀ value that showed 50% protection against Fe²⁺-induced free radical neurotoxicity (Table 1), showing that potent neuroprotective effects of BAS, CBAS, FBAS, TBAS, PBAS, MBAS, NPAA, NPPAA, 2-Hydroxy-TTBA, and 2-Hydroxy-TPEA as compared to vitamin E.

TABLE 1
BLOCKADE OF Fe²⁺-INDUCED FREE
RADICAL NEUROTOXICITY BY VITAMIN E, TROLOX,
BENZYLAMINOSALICYLIC ACID DERIVATIVES
AND A TETRAFLUOROBENZYL DERIVATIVE.

| Drug | IC₅₀ (μM) |
|---|---|
| BAS | 1.24 |
| NBAS | 1.9 |
| CBAS | 0.2 |
| TBAS | 0.31 |
| MBAS | 1.42 |
| FBAS | 0.3 |
| PBAS | 0.1 |
| NPAA | 0.27 |
| NPPAA | 0.20 |
| 2-Hydroxy-TTBA | 0.11 |
| 2-Hydroxy-TPEA | 0.099 |
| Trolox | 3.34 |
| Vitamin E | 22.03 |

However, concurrent administration of 10 mM Li⁺, which was shown to attenuate apoptosis (Kang et al, 2003), did not attenuate Fe²⁺- or BSO-induced free radical neurotoxicity (FIG. 1C).

### EXAMPLE 3

Prevention of Neuronal Cell Apoptosis by Li⁺

Cortical cell cultures containing neurons and glia at 10-12 days in vitro (DIV 10-12) were exposed to 20 μM cyclosporine A (CsA) or 10 nM caliculin A (cal A). Neurons underwent widespread apoptosis 24 hr later as previously reported (McDonald et al., 1996; Ko et al., 2000). Concurrent administration of Li⁺ dose-dependently attenuated neuronal cell apoptosis at doses of 3-30 mM (FIG. 2A). Cyclosporine A-induced neuronal cell apoptosis was not attenuated by inclusion of vitamin E, 2-hydroxy-TTBA, or 2-hydroxy-TPEA (FIG. 2B). This implies that Li⁺ and the neuroprotective drugs (vitamin E, trolox, BAS, CBAS, FBAS, TBAS, PBAS, MBAS, NPAA, NPPAA, 2-Hydroxy-TTBA, and 2-Hydroxy-TPEA) selectively prevent neuronal cell apoptosis and free radical-mediated necrosis, respectively.

### EXAMPLE 4

### Enhanced Prevention of Neuronal Cell Death and Motor Performance Deficit in Transgenic Mouse Model of ALS (G93A Mouse) by Combination of Both 2-Hydroxy-TTBA and Lithium

(4-1) Onset of Oxidative Stress Prior to Motor Neuron Degeneration in G93A Transgenic Mice

Levels of oxidative stress were first examined in the spinal cord from wild type and transgenic mice before behavioral deficit and motor neuron degeneration were observed. Marked oxidative stress was observed in the motor neurons in the lumbar ventral horn from G93A transgenic mice compared to the wild type at ages of 8 weeks as shown by increased immunoreactivity to nitrotyrosine antibody (FIG. 3A). Fluorescence intensity of oxidized MitoTracker CM-H₂XRos was also increased in the spinal motor neurons from the transgenic mice, suggesting that the spinal motor neurons are accompanied by accumulation of protein oxidation and by free radicals. Similar levels of nitrotyrosine immunoreactivity and mitochondrial free radicals were observed in the dorsal horn neurons and white matter. Analysis of nitrotyrosine immunoreactivity showed that oxidative stress was increased up to 3-fold in the motor neurons from the transgenic mice compared to the wild type at ages of 4 weeks (FIG. 3B). Levels of nitrotyrosine were peaked to 4-fold at 8 weeks of age and then declined over 14 weeks of age. Neuronal death was slightly observed in the ventral horn from the transgenic mice at 8 weeks of age when oxidative stress was peaked (FIG. 3C). After then, neuronal death was gradually observed until the animals would die. This implies that G93AA transgenic mice produce oxidative stress selectively in the motor neurons at the early ages, which may in turn cause neurodegeneration in the lumbar ventral horn.

(4-2) Activation of Fas-Mediated Apoptosis Signaling Pathway in G93A Transgenic Mice

Fas ligand (FasL)-mediated apoptosis plays a role in neuronal death in neurodegenerative diseases including Alzheimer's disease, Parkinson's disease, and (Morishima et al., 2001, Su et al, 2003; Hartman et al., 2002). It is conceivable to reason that the Fas signaling pathway contributes to apoptosis of the motor neurons in G93A transgenic mice. Expression and interaction of Fas and its cytoplasmic adaptor protein FADD were found to have increased in the lumbar spinal cord from the transgenic mice at 12 weeks of age compared to the wild type (FIG. 4A). Immunohistochemistry with Fas antibody revealed that levels of Fas were increased primarily in the spinal motor neurons from G93A mice (FIG. 4B). The death-inducing signaling complex was followed by activation of caspase-8, possibly through the autoproteolytic processing of procaspase-8, and caspase-3 (FIG. 4C). The active form of caspase-3 was observed primarily in the motor neurons in the lumbar spinal cord from G93A mice (FIG. 4D). This suggests that that Fas, FADD, caspase-8, and caspase-3 are activated in the spinal motor neurons to mediate subsequent neuronal apoptosis in the ALS mice at ages of 12 weeks. The activation pattern of the Fas-signaling molecules disappeared at ages of 16 weeks when most motor neurons died.

(4-3) 2-Hydroxy-TTBA and Li⁺ Prevent Oxidative Stress and Apoptosis in Cortical Cell Cultures and in G93A Transgenic Mice, Respectively

Additional experiments were performed to examine if targeting both neuronal cell necrosis and apoptosis would result in synergic neuroprotection in G93AA transgenic mice. Oxidative stress was induced by exposure of cortical cell cultures containing neurons and glia to OH radial-producing transition metal Fe²⁺ or glutathione-depleting agent buthionine sulfoximine (BSO) that were shown to cause widespread neuronal cell necrosis within 24 hr. Fe²⁺- and BSO-induced neuronal death was completely blocked by concurrent administration of 2-hydroxy-5-(2,3,5,6-tetrafluoro-4-trifluoromethyl-benzylamino)-benzoic acid (2-hydroxy-TTBA) even at a submicromolar concentration (FIG. 5A). The neuroprotective effect of 2-hydroxy-TTBA against Fe²⁺-induced oxidative neuronal death was 220 times higher than vitamin E. The oxidative neuronal death was not attenuated by addition of lithium ion (Li⁺), a mood-stabilizing agent which was reported to selectively prevent neuronal cell apoptosis without protective effects against excitotoxic neuronal cell necrosis (Kang et al., 2003; Chuang et al., 2002). Neuronal cell apoptosis was induced by serum deprivation in neuron-rich cortical cell cultures as reported, which was prevented by addition of 5 mM Li⁺ as well as zVADfmk, a broad spectrum inhibitor of caspases (FIG. 5B). Serum deprivation-induced neuronal cell apoptosis was not attenuated by addition of 2-Hydroxy-TTBA. Additional experiments were performed to examine if Li⁺ would prevent Fas signaling pathway. Fas-FADD interaction was observed in neuron-rich cortical cell cultures deprived of serum for 8 hr, which was blocked by addition of Li⁺, but not by 2-Hydroxy-TTBA (FIG. 5C). This implies that 2-Hydroxy-TTBA and Li⁺ blocks oxidative neuronal cell necrosis and apoptosis, respectively.

G93A transgenic mice received oral administration of 2-hydroxy-TTBA (30 mg/kg/d) in the diet from 8 weeks of age. The oral administration of 2-Hydroxy-TTBA blocked nitrotyrosine, and mitochondrial free radical increased in the lumbar spinal motor neurons at 10 weeks of age compared to the wild type (FIGS. 5D & 5E). The administration of 2-Hydroxy-TTBA slightly attenuated levels of Fas, FADD, and cleaved caspase-8 and caspase-3 increased in the lumbar spinal cord from G93A transgenic mice at 12 weeks of age (FIG. 5F). Oral administration of Li⁺ (200 mg/kg/d) in the diet completely blocked the Fas pathway induced in the spinal cord from G93A mice. Thus, cell necrosis and Fas-mediated apoptosis induced in G93A mice can be prevented by oral administration of 2-hydroxy-TTBA and Li⁺.

(4-4) 2-Hydroxy-TTBA and Lithium Synergically Delay Onset and Progression of Motor Deficit in G93A Transgenic Mice

G93A transgenic mice revealed body weight loss down to 58% of the wild type at 18 weeks of age (FIG. 6A). The oral administration of 2-Hydroxy-TTBA or Li⁺ from 12 weeks of age alleviated weight loss to 41 and 53% of the wild type. The weight loss was further reduced to 32% by co-administration of 2-Hydroxy-TTBA and Li⁺. G93A transgenic mice fed with 2-Hydroxy-TTBA or Li⁺ in the diet showed better motor performance than the vehicle-treated control from 11 weeks to 18 weeks (FIG. 6B-6D). Onset of PaGE deficits or Rotarod deficits and mortality of ALS transgenic mice were analyzed, mean±SEM (n=13 per each group) a, p<0.01 compared to vehicle; b, p<0.05 between 2-hydroxy TTBA (or Li) alone and combination of 2-hydroxy TTBA and Li. Extension reflex, motor strength, and coordination were all improved in the transgenic ALS mice treated with either 2-Hydroxy-TTBA or Li⁺. Onset of PaGE deficiency was 104 days in vehicle-treated G93A control mice and delayed to 114.1 and 113.3 days in G93A mice treated with 2-Hydroxy-TTBA and Li⁺, respectively (Table 2).

TABLE 2
DELAYED ONSET OF MOTOR DEFICIT AND MORTALITY
OF ALS MICE TREATED WITH 2-HYDROXY-TTBA AND/OR
LITHIUM (MEAN ± SED, N = 13 PER EACH GROUP)

| | | | | 2-Hydroxy- |
|---|---|---|---|---|
| | Vehicle | 2-Hydroxy-TTBA | Li | TTBA + Li |
| Onset from PaGE | 104 ± 2.70 | 114.1^{a} ± 2.02 | 113.3^{a} ± 2.28 | 127.6^{a,b} ± 7.39 |
| Onset from Rotarod | 98.7 ± 3.30 | 112.3^{a} ± 2.89 | 114.7^{a} ± 2.23 | 121.5^{a,b} ± 4.67 |
| Mortality | 125.3 ± 2.10 | 143.8^{a} ± 2.83 | 137.2^{a} ± 2.20 | 152.1^{a,b} ± 5.87 |

^{a}P < 0.01 compared with vehicle group
^{b}P < 0.05 compared with 2-Hydroxy-TTBA and lithium group

As shown in Table 2 and FIG. 7A, the onset was further delayed to 127.6 days in G93A mice treated with both 2-Hydroxy-TTBA and Li⁺. In rotarod test, onset of impaired motor performance was 98.7 days in vehicle-treated control group. The onset was 112.3 and 114.7 days in G93A mice administered with 2-Hydroxy-TTBA and Li⁺, respectively, which was further delayed to 121.5 days following co-administration of both 2-Hydroxy-TTBA and Li⁺.

Administration of 2-Hydroxy-TTBA and Li⁺ extended survival from 125.6 days to 143.8 and 137.2 days in G93A transgenic mice (Table 2, FIG. 7B). Survival was further extended to 152.1 days in G93A mice administrated with both 2-Hydroxy-TTBA and Li⁺. Finally, neuroprotective effects of 2-Hydroxy-TTBA or Li⁺ were examined in the ventral motor neurons from the lumbar spinal cord at 16 weeks of age. In the control G93A mice, motor neurons underwent widespread degeneration up to 74% (FIGS. 7C & 7D). Degeneration of motor neurons was reduced to 57 and 58% in G93A mice treated with 2-Hydroxy-TTBA and Li⁺, respectively. Neuronal loss was further reduced to 17% in G93A mice treated with combination of 2-Hydroxy-TTBA and Li⁺.

### EXAMPLE 5

### The Effect of 2-Hydroxy-TPEA and Lithium on Amyloid Beta Production in TAPP Transgenic Mice

(5-1) Reduction of Amyloid Plaque Burden in 14.5 Month TAPP Mouse Model

3.5 month-old wt-TAPP and tt-TAPP (Tg2576+P301 L: double tg) mice were fed chow alone (saline only), or containing 25 mg/kg/day of 2-hydroxy-TPEA, or 25 mg/kg/day of 2-hydroxy-TPEA+300 mg/kg/day of lithium carbonate, for 11 months before being sacrificed (3.5M˜14.5 Month).

18˜20 μm brain sections stained 1% Thioflavin-S for 5 min and observed under fluorescence microscope system.

As a quantitative analysis of amyloid burden, co-treatment with 25 mg/kg/d of 2-hydroxy-TPEA and 300 mg/kg/d of lithium carbonate caused a significant 42% reduction in plaque burden (FIG. 8(B)).

(5-2) Reduction of SDS-Insoluble Aβ42 Levels in Drug-Treated TAPP Mouse Model

3.5 month-old wt-TAPP and tt-TAPP (Tg2576+P301L: double tg) mice were fed chow alone, or containing 25 mg/kg/day of 2-hydroxy-TPEA, or 25 mg/kg/day of 2-hydroxy-TPEA+300 mg/kg/day of lithium carbonate for 11 months before being sacrificed (3.5M˜14.5 Month).

SDS-insoluble Aβ42 levels were analyzed by colorimetric sandwich ELISA kit (BIOSOURCE, Camarillo, Calif.).

Co-treatment group with 25 mg/kg/d of 2-hydroxy-TPEA and 300 mg/kg/d of lithium carbonate group (44%) showed a better reduction in SDS-insoluble Aβ42 levels compared to 2-hydroxy-TPEA only group (10%) (FIG. 8(C)).

As described above, the combination of cell necrosis inhibitors and lithium of the present invention can effectively be used to treat neurological diseases or ocular diseases.

Examples of concrete diseases applicable with the combination of the present invention are described as follows. However, the scope of the present invention is not limited to the diseases described below.

### APPLICATION EXAMPLE 1

### Lou Gehrig Disease (or Amyotrophic Lateral Sclerosis)

Lou Gehrig Disease is named amyotrophic lateral sclerosis (ALS) or motor neuron disease, and the progressive degeneration of upper and lower motor neurons is the pathological hallmark of this disease. Many hypotheses have been put forward to account for the selective death of motor neurons in ALS.

ALS patients show increased levels of extracellular glutamate and loss of glutamate transporter GLT-1. Administration of glutamate receptor agonists into the spinal cord mimicked pathological changes in the spinal cord of ALS patients (Rothstein J D et al., 1995; Ikonomidou C et al., 1996).

The recent discovery of mutations affecting the superoxide dismutase (SOD) gene has given impetus to research on the role of oxidative stress in the pathogenesis of familial ALS (Robberecht W, 2000). Nonetheless, evidence shows that there is abnormal oxidative damage to proteins in postmortem samples from ALS patients. Post-mortem studies in ALS patients demonstrated increased nitrotyrosine immunoreactivity and total protein carbonylation in spinal motor neurons (Abe K et al., 1995; Shaw P J et al., 1995).

Recently, interest has been generated by the possibility that a mechanism of programmed cell death, termed apoptosis, is responsible for the motor neuron degeneration in ALS (Sathasivam S et al., 2001).

Therefore, a combination of the present invention can be used as therapeutic drugs for ALS.

### APPLICATION EXAMPLE 2

### Alzheimer's Disease

Alzheimer's disease is the most common form of adult onset dementia. Alzheimer's disease is characterized as the presence of the neurofibrillary tangles (NFT), amyloid plaques and neuronal death.

The direct evidence supporting increased oxidative stress in AD is: (1) increased brain Fe, Al, and Hg in AD, capable of stimulating free radical generation; (2) increased lipid peroxidation in AD brain; (3) increased protein and DNA oxidation in the AD brain (Olanow C W et al., 1994; Markesbery W R, 1997).

Also, a low- to moderate-affinity uncompetitive N-methyl-D-aspartate receptor antagonist, memantine, has been shown to improve learning and memory in several pharmacological models of AD, suggesting that NMDA antagonist has therapeutic potential in AD (Minkeviciene R et al., 2004).

Several studies have shown the activation of caspase-3 or caspase-9 during apoptosis in Alzheimer's disease (Kang H J et al., 2005; Chong Z Z et al., 2005).

Therefore, the combination of the present invention showing protective effect against cell necrosis and apoptosis can be used as therapeutic drugs for Alzheimer's disease.

### APPLICATION EXAMPLE 3

### Parkinson's Disease (PD)

Parkinson's Disease (PD), the prototypic movement disorder, is characterized clinically by tremor, rigidity, bradykinesia and postural instability and diagnosed pathologically by a selective death of dopaminergic neurons in the substantia nigra.

In PD patients, oxidative stress has been proved as a main mechanism of dopaminergic neuronal cell death, and the increased production of lipid peroxidation and ROS and the decreased GSH contents has been reported, suggesting that oxidative stress plays a causative role in neuronal death in PD (Sriram K et al., 1997; Wu D C et al., 2003).

Also, several antagonists of NMDA receptors protect dopaminergic neurons from the dopaminergic neurotoxin MPTP (1-methyl-4-phenyl-1,2,3,6-tetrahydropyridine) (Brouillet E and Beal M F, 1993).

Many in vivo studies have shown that there is some evidence for the occurrence of apoptosis in the Parkinsonian substantia. For example, there is increased neuronal expression of caspases (Hartmann A et al., 2000 and 2001) in animal model of Parkinson's Disease, suggesting that these cells are undergoing apoptosis.

Therefore, the combination of the present invention showing protective effect against cell necrosis and apoptosis can be used as therapeutic drugs for Parkinson's disease.

### APPLICATION EXAMPLE 4

### Huntington's Disease (HD)

Huntington's disease (HD) is a progressive neurodegenerative disease predominantly affecting small- and medium-sized interneurons in the striata.

These pathological features of HD are observed in vivo and in vitro following administration of NMDA receptor agonists, raising the possibility that NMDA receptor-mediated neurotoxicity contributes to selective neuronal death in HD (Koh J Y et al., 1986; Beal M F et al., 1986).

Strialtal projection neurons are highly vulnerable to apoptosis in HD. Recent data have shown that there is increased expression of cytochrome C and caspase-9 in HD (Kiechle T et al., 2002) and also many TUNEL-positive cells accompanied with weak caspase-3 immunoreactivity in severely affected HD brains, suggests that neuronal apoptosis plays a role in HD (Vis J C et al., 2005).

Since evidence is being accumulated that oxidative stress, such as mitochondrial dysfunction and generation of ROS, causes neuronal death observed in HD, it is possible that the drugs inhibiting ROS are used for therapy of HD (Perez-Severiano F et al., 2003; Rosenstock T R et al., 2004).

Therefore, the combination of the present invention showing protective effect against cell necrosis and apoptosis can be used as therapeutic drugs for HD.

### APPLICATION EXAMPLE 5

### Stroke

Stroke is a sudden problem affecting the blood vessels of the brain, and interrupted blood supply to brain or stroke induces neuronal death primarily through overactivation of glutamate receptor. It has been well documented that NMDA receptor antagonists decrease the neuronal cell death by ischemic stroke [Simon R P et al., 1984].

Also, when brain hypoxic ischemia occurs, mitochondrial electron transport system can be injured, so ROS production increases. Increased production of ROS is capable of causing neuronal death through lipid peroxidation, DNA oxidation or protein oxidation. Some antioxidants showed efficiency in animal models of hypoxic ischemia (Yamaguchi T et al., 1998).

It has also been reported that apoptosis is main mechanism of neuronal death following hypoxic ischemia. Markers of neuronal apoptotic cell death were observed in regions with hypoxic ischemia (Hu X et al., 2002).

Therefore, the combination of the present invention showing protective effect against cell necrosis and apoptosis can be used as therapeutic drugs for stroke.

### APPLICATION EXAMPLE 6

### Traumatic Brain Injury (TBI) and Traumatic Spinal Cord Injury (TSCI)

Excitotoxins are closely related to the degeneration of neuronal cells following traumatic brain injury (TBI) and traumatic spinal cord injury (TSCI). It has been reported that NMDA receptor antagonists decrease the neuronal death following TBI and TSCI (Faden Al et al., 1988; Okiyama K et al., 1997).

Traumatic injuries to spinal cord or brain cause tissue damage, in part by initiating reactive biochemical changes. Numerous studies have provided considerable support for lipid peroxidation reactions, Ca²⁺ influx, and disruption of membrane in the TBI and TSCI and anti-oxidants also inhibit tissue damage following TBI and TSCI (Faden Al and Salzman S, 1992; Juurlink B H and Paterson P G, 1998).

Recent evidence provides that special caspases expression can be found in the TBI and TSCI and also inhibition of caspase has therapeutic in the treatment of TBI and TSCI (Clark R S et al., 2000; Li M et al., 2000; Keane R W et al., 2001).

Therefore, the combination of the present invention showing protective effect against cell necrosis and apoptosis can be used as therapeutic drugs for traumatic Spinal Cord injuries.

### APPLICATION EXAMPLE 7

### Glaucoma, Diabetic Retinopathy or Macular Degeneration

In glaucoma, the increased intraocular pressure blocks blood flow into retina and causes retinal hypoxia. The degeneration of retina cells can also occur through excitotoxicity and the increased generation of reactive oxygen species during reperfusion and also hypoxia lead to apoptosis (Osborne N N et al., 1999; Hartwick A T, 2001; Nickells R W, 1999; Tempestini A et al., 2003). Recent studies have demonstrated that antioxidants may be a new therapeutic tool to prevent ocular diseases (Neufeld A H et al., 2002; Richer S et al., 2004).

Also, increasing amounts of evidence suggest that neurodegeneration in diabetic retinopathy and macular degeneration relates to excitotoxicity, oxidative damage and apoptosis (Lieth E et al., 2000; Moor P et al., 2001; Simonelli F et al., 2002; Barber A J, 2003; Joussen A M et al., 2003).

Therefore, the combination of the present invention showing protective effect against cell necrosis and apoptosis can be used as therapeutic drugs for ocular diseases such as glaucoma, diabetic retinopathy and macular degeneration.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention.

What is claimed is:

1. A method for treating neuronal death in neurological disease or ocular disease in a human or animal, which comprises administering to the human or animal in need thereof a therapeutically effective amount of a cell necrosis inhibitor and concomitantly or sequentially administering a therapeutically effective amount of lithium or a pharmaceutically acceptable salt thereof.

2. The method of claim 1, wherein the neurological disease is selected from amyotrophic lateral sclerosis (ALS, Lou Gehrig's disease), Alzheimer's disease, Parkinson's disease, Huntington's disease, stroke, traumatic brain injury, and spinal cord injury.

3. The method of claim 1, wherein the ocular disease is selected from glaucoma, diabetic retinopathy and macular degeneration.

4. The method of claim 1, wherein the cell necrosis inhibitor is at least one selected from:
(i) benzylaminosalicylic acid derivatives of the following formula (I) or pharmaceutically acceptable salts thereof and
(ii) tetrafluorobenzyl derivatives of the following formula (II) or pharmaceutically acceptable salts thereof: 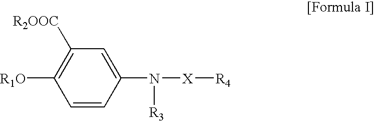
wherein,
X is CO, SO₂ or (CH₂)ₙ, wherein n is an integer from 1 to 5;
R₁ is hydrogen, alkyl or alkanoyl;
R₂ is hydrogen or alkyl;
R₃ is hydrogen or an acetoxy group; and
R₄ is a phenyl group which is unsubstituted or substituted with one or more of nitro, halogen, haloalkyl, and C₁-C₅ alkoxy; 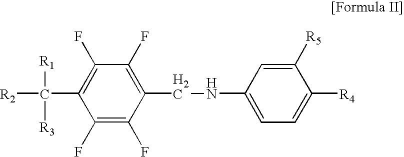
wherein,
R₁, R₂ and R₃ are independently hydrogen or halogen;
R₄ is hydroxy, alkyl, alkoxy, halogen, alkoxy substituted with halogen, alkanoyloxy or nitro; and
R₅ is carboxyl acid, ester having C₁-C₄ alkyl, carboxyamide, sulfonic acid, halogen or nitro.

5. The method of claim 4, wherein the benzylaminosalicylic acid derivative is at least one selected from:
5-benzylaminosalicylic acid,
5-(4-nitrobenzyl)aminosalicylic acid,
5-(4-chlorobenzyl)aminosalicylic acid,
5-(4-trifluoromethylbenzyl)aminosalicylic acid,
5-(4-fluorobenzyl)aminosalicylic acid,
5-(4-methoxybenzyl)aminosalicylic acid,
5-(4-pentafluorobenzyl)aminosalicylic acid,
5-(4-nitrobenzyl)amino-2-hydroxy ethylbenzoate,
5-(4-nitrobenzyl)-N-acetylamino-2-hydroxy ethylbenzoate,
5-(4-nitrobenzyl)-N-acetylamino-2-acetoxy ethylbenzoate,
5-(4-nitrobenzoyl)aminosalicylic acid,
5-(4-nitrobenzenesulfonyl)aminosalicylic acid,
5-[2-(4-nitrophenyl)ethyl]aminosalicylic acid,
5-[3-(4-nitrophenyl)-n-propyl]aminosalicylic acid, and
2-hydroxy-5-(2-(4-trifluoromethyl-phenyl)ethylamino)-benzoic acid.

6. The method of claim 5, wherein the benzylaminosalicylic acid derivative is 2-hydroxy-5-(2-(4-trifluoromethyl-phenyl)ethylamino)-benzoic acid.

7. The method of claim 4, wherein the tetrafluorobenzyl derivative is at least one selected from:
2-hydroxy-5-(2,3,5,6-tetrafluoro-4-trifluoromethyl-benzylamino)-benzoic acid,
2-nitro-5-(2,3,5,6-tetrafluoro-4-trifluoromethyl-benzylamino)-benzoic acid,
2-chloro-5-(2,3,5,6-tetrafluoro-4-trifluoromethyl-benzylamino)-benzoic acid,
2-bromo-5-(2,3,5,6-tetrafluoro-4-trifluoromethyl-benzylamino)-benzoic acid,
2-hydroxy-5-(2,3,5,6-tetrafluoro-4-methyl-benzylamino)-benzoic acid,
2-methyl-5-(2,3,5,6-tetrafluoro-4-trifluoromethyl-benzylamino)-benzoic acid,
2-methoxy-5-(2,3,5,6-tetrafluoro-4-trifluoromethyl-benzylamino)-benzoic acid,
5-(2,3,5,6-tetrafluoro-4-trifluoromethyl-benzylamino)-2-trifluoromethoxy benzoic acid,
2-nitro-4-(2,3,5,6-tetrafluoro-4-trifluoromethyl-benzylamino)phenol,
2-chloro-4-(2,3,5,6-tetrafluoro-4-trifluoromethyl-benzylamino)-phenol,
2-hydroxy-5-(2,3,5,6-tetrafluoro-4-trifluoromethyl-benzylamino)-benzamide,
2-hydroxy-5-(2,3,5,6-tetrafluoro-4-trifluoromethyl-benzylamino)-benzenesulfonic acid,
methyl 2-hydroxy-5-(2,3,5,6-tetrafluoro-4-trifluoromethyl-benzylamino)-benzoate,
2-ethanoyloxy-5-(2,3,5,6-tetrafluoro-4-trifluoromethyl-benzylamino)-benzoic acid,
2-propanoyloxy-5-(2,3,5,6-tetrafluoro-4-trifluoromethyl-benzylamino)-benzoic acid, and
2-cyclohexan carbonyloxy-5-(2,3,5,6-tetrafluoro-4-trifluoromethyl-benzylamino)-benzoic acid.

8. The method of claim 7, wherein the tetrafluorobenzyl derivative is 2-hydroxy-5-(2,3,5,6-tetrafluoro-4-trifluoromethyl-benzylamino)-benzoic acid.

9. A pharmaceutical formulation for treating neuronal death in neurological disease or ocular disease in a human or animal, which comprises a therapeutically effective amount of a cell necrosis inhibitor and a therapeutically effective amount of lithium or a pharmaceutical acceptable salt thereof.

10. The pharmaceutical formulation of claim 9, wherein the neurological disease is selected from amyotrophic lateral sclerosis (ALS, Lou Gehrig's disease), Alzheimer's disease, Parkinson's disease, Huntington's disease, stroke, traumatic brain injury, and spinal cord injury.

11. The pharmaceutical formulation of claim 9, wherein the ocular disease is selected from glaucoma, diabetic retinopathy and macular degeneration.

12. The pharmaceutical formulation of claim 9, wherein the cell necrosis inhibitor is at least one selected from:
(i) benzylaminosalicylic acid derivatives of the following formula (I) or pharmaceutically acceptable salts thereof and
(ii) tetrafluorobenzyl derivatives of the following formula (II) or pharmaceutically acceptable salts thereof: 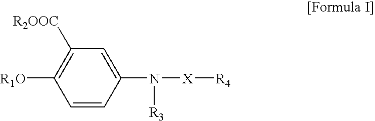
wherein,
X is CO, SO₂ or (CH₂)ₙ, wherein n is an integer from 1 to 5;
R₁ is hydrogen, alkyl or alkanoyl;
R₂ is hydrogen or alkyl;
R₃ is hydrogen or an acetoxy group; and
R₄ is a phenyl group which is unsubstituted or substituted with one or more of nitro, halogen, haloalkyl, and C₁-C₅ alkoxy; 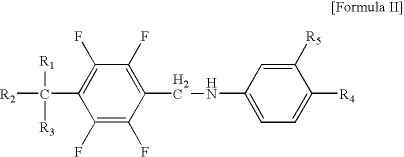
wherein,
R₁, R₂ and R₃ are independently hydrogen or halogen;
R₄ is hydroxy, alkyl, alkoxy, halogen, alkoxy substituted with halogen, alkanoyloxy or nitro; and
R₅ is carboxyl acid, ester having C₁-C₄ alkyl, carboxyamide, sulfonic acid, halogen or nitro.

13. The pharmaceutical formulation of claim 12, wherein the benzylaminosalicylic acid derivative is at least one selected from:
5-benzylaminosalicylic acid,
5-(4-nitrobenzyl)aminosalicylic acid,
5-(4-chlorobenzyl)aminosalicylic acid,
5-(4-trifluoromethylbenzyl)aminosalicylic acid,
5-(4-fluorobenzyl)aminosalicylic acid,
5-(4-methoxybenzyl)aminosalicylic acid,
5-(4-pentafluorobenzyl)aminosalicylic acid,
5-(4-nitrobenzyl)amino-2-hydroxy ethylbenzoate,
5-(4-nitrobenzyl)-N-acetylamino-2-hydroxy ethylbenzoate,
5-(4-nitrobenzyl)-N-acetylamino-2-acetoxy ethylbenzoate,
5-(4-nitrobenzoyl)aminosalicylic acid,
5-(4-nitrobenzenesulfonyl)aminosalicylic acid,
5-[2-(4-nitrophenyl)ethyl]aminosalicylic acid,
5-[3-(4-nitrophenyl)-n-propyl]aminosalicylic acid, and
2-hydroxy-5-(2-(4-trifluoromethyl-phenyl)ethylamino)-benzoic acid.

14. The pharmaceutical formulation of claim 13, wherein the benzylaminosalicylic acid derivative is 2-hydroxy-5-(2-(4-trifluoromethyl-phenyl)ethylamino)-benzoic acid.

15. The pharmaceutical formulation of claim 12, wherein the tetrafluorobenzyl derivatives is at least one selected from:
2-hydroxy-5-(2,3,5,6-tetrafluoro-4-trifluoromethyl-benzylamino)-benzoic acid,
2-nitro-5-(2,3,5,6-tetrafluoro-4-trifluoromethyl-benzylamino)-benzoic acid,
2-chloro-5-(2,3,5,6-tetrafluoro-4-trifluoromethyl-benzylamino)-benzoic acid,
2-bromo-5-(2,3,5,6-tetrafluoro-4-trifluoromethyl-benzylamino)-benzoic acid,
2-hydroxy-5-(2,3,5,6-tetrafluoro-4-methyl-benzylamino)-benzoic acid,
2-methyl-5-(2,3,5,6-tetrafluoro-4-trifluoromethyl-benzylamino)-benzoic acid,
2-methoxy-5-(2,3,5,6-tetrafluoro-4-trifluoromethyl-benzylamino)-benzoic acid,
5-(2,3,5,6-tetrafluoro-4-trifluoromethyl-benzylamino)-2-trifluoromethoxy benzoic acid,
2-nitro-4-(2,3,5,6-tetrafluoro-4-trifluoromethyl-benzylamino)phenol,
2-chloro-4-(2,3,5,6-tetrafluoro-4-trifluoromethyl-benzylamino)-phenol,
2-hydroxy-5-(2,3,5,6-tetrafluoro-4-trifluoromethyl-benzylamino)-benzamide,
2-hydroxy-5-(2,3,5,6-tetrafluoro-4-trifluoromethyl-benzylamino)-benzenesulfonic acid,
methyl 2-hydroxy-5-(2,3,5,6-tetrafluoro-4-trifluoromethyl-benzylamino)-benzoate,
2-ethanoyloxy-5-(2,3,5,6-tetrafluoro-4-trifluoromethyl-benzylamino)-benzoic acid,
2-propanoyloxy-5-(2,3,5,6-tetrafluoro-4-trifluoromethyl-benzylamino)-benzoic acid, and
2-cyclohexan carbonyloxy-5-(2,3,5,6-tetrafluoro-4-trifluoromethyl-benzylamino)-benzoic acid.

16. The pharmaceutical formulation of claim 15, wherein the tetrafluorobenzyl derivatives is 2-hydroxy-5-(2,3,5,6-tetrafluoro-4-trifluoromethyl-benzylamino)-benzoic acid.

17. The compound 2-hydroxy-5-(2-(4-trifluoromethyl-phenyl)ethylamino)-benzoic acid or a pharmaceutically acceptable salt thereof.

18. The compound of claim 17 and a pharmaceutically acceptable carrier or diluent.

19. A kit for treating neuronal death in neurological disease or ocular disease in a human or animal, which comprises a therapeutically effective amount of a cell necrosis inhibitor and a therapeutically effective amount of lithium or a pharmaceutical acceptable salt thereof.

20. The kit of claim 19, wherein the neurological disease is selected from amyotrophic lateral sclerosis (ALS, Lou Gehrig's disease), Alzheimer's disease, Parkinson's disease, Huntington's disease, stroke, traumatic brain injury, and spinal cord injury.

21. The kit of claim 19, wherein the ocular disease is selected from glaucoma, diabetic retinopathy and macular degeneration.

22. The kit of claim 19, wherein the cell necrosis inhibitor is at least one selected from:
(i) benzylaminosalicylic acid derivatives of the following formula (I) or pharmaceutically acceptable salts thereof and
(ii) tetrafluorobenzyl derivatives of the following formula (II) or pharmaceutically acceptable salts thereof: 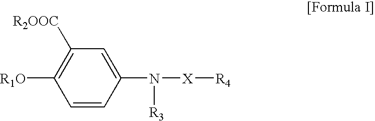
wherein,
X is CO, SO₂ or (CH₂)ₙ, wherein n is an integer from 1 to 5;
R₁ is hydrogen, alkyl or alkanoyl;
R₂ is hydrogen or alkyl;
R₃ is hydrogen or an acetoxy group; and
R₄ is a phenyl group which is unsubstituted or substituted with one or more of nitro, halogen, haloalkyl, and C₁-C₅ alkoxy; 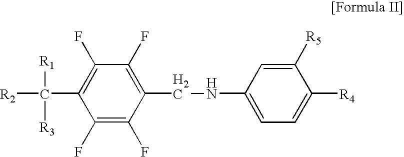
wherein,
R₁, R₂ and R₃ are independently hydrogen or halogen;
R₄ is hydroxy, alkyl, alkoxy, halogen, alkoxy substituted with halogen, alkanoyloxy or nitro; and
R₅ is carboxyl acid, ester having C₁-C₄ alkyl, carboxyamide, sulfonic acid, halogen or nitro.

23. The kit of claim 22, wherein the benzylaminosalicylic acid derivative is at least one selected from:
5-benzylaminosalicylic acid,
5-(4-nitrobenzyl)aminosalicylic acid,
5-(4-chlorobenzyl)aminosalicylic acid,
5-(4-trifluoromethylbenzyl)aminosalicylic acid,
5-(4-fluorobenzyl)aminosalicylic acid,
5-(4-methoxybenzyl)aminosalicylic acid,
5-(4-pentafluorobenzyl)aminosalicylic acid,
5-(4-nitrobenzyl)amino-2-hydroxy ethylbenzoate,
5-(4-nitrobenzyl)-N-acetylamino-2-hydroxy ethylbenzoate,
5-(4-nitrobenzyl)-N-acetylamino-2-acetoxy ethylbenzoate,
5-(4-nitrobenzoyl)aminosalicylic acid,
5-(4-nitrobenzenesulfonyl)aminosalicylic acid,
5-[2-(4-nitrophenyl)ethyl]aminosalicylic acid,
5-[3-(4-nitrophenyl)-n-propyl]aminosalicylic acid, and
2-hydroxy-5-(2-(4-trifluoromethyl-phenyl)ethylamino)-benzoic acid.

24. The kit of claim 23, wherein the benzylaminosalicylic acid derivative is 2-hydroxy-5-(2-(4-trifluoromethyl-phenyl)ethylamino)-benzoic acid.

25. The kit of claim 22, wherein the tetrafluorobenzyl derivatives is at least one selected from:
2-hydroxy-5-(2,3,5,6-tetrafluoro-4-trifluoromethyl-benzylamino)-benzoic acid,
2-nitro-5-(2,3,5,6-tetrafluoro-4-trifluoromethyl-benzylamino)-benzoic acid,
2-chloro-5-(2,3,5,6-tetrafluoro-4-trifluoromethyl-benzylamino)-benzoic acid,
2-bromo-5-(2,3,5,6-tetrafluoro-4-trifluoromethyl-benzylamino)-benzoic acid,
2-hydroxy-5-(2,3,5,6-tetrafluoro-4-methyl-benzylamino)-benzoic acid,
2-methyl-5-(2,3,5,6-tetrafluoro-4-trifluoromethyl-benzylamino)-benzoic acid,
2-methoxy-5-(2,3,5,6-tetrafluoro-4-trifluoromethyl-benzylamino)-benzoic acid,
5-(2,3,5,6-tetrafluoro-4-trifluoromethyl-benzylamino)-2-trifluoromethoxy benzoic acid,
2-nitro-4-(2,3,5,6-tetrafluoro-4-trifluoromethyl-benzylamino)phenol,
2-chloro-4-(2,3,5,6-tetrafluoro-4-trifluoromethyl-benzylamino)-phenol,
2-hydroxy-5-(2,3,5,6-tetrafluoro-4-trifluoromethyl-benzylamino)-benzamide,
2-hydroxy-5-(2,3,5,6-tetrafluoro-4-trifluoromethyl-benzylamino)-benzenesulfonic acid,
methyl 2-hydroxy-5-(2,3,5,6-tetrafluoro-4-trifluoromethyl-benzylamino)-benzoate,
2-ethanoyloxy-5-(2,3,5,6-tetrafluoro-4-trifluoromethyl-benzylamino)-benzoic acid,
2-propanoyloxy-5-(2,3,5,6-tetrafluoro-4-trifluoromethyl-benzylamino)-benzoic acid, and
2-cyclohexan carbonyloxy-5-(2,3,5,6-tetrafluoro-4-trifluoromethyl-benzylamino)-benzoic acid.

26. The kit of claim 25, wherein the tetrafluorobenzyl derivatives is 2-hydroxy-5-(2,3,5,6-tetrafluoro-4-trifluoromethyl-benzylamino)-benzoic acid.
